(19) United States
(12) Patent Application Publication
(10) Pub. No.: US 2007/0059347 A1
(43) Pub. Date: Mar. 15, 2007

(54) ANTIBACTERIAL FIBER ASSEMBLY AND PRODUCTION METHOD AND USES THEREOF

(75) Inventors: Hidefumi Suzuki, Sodegaura-shi, Chiba (JP); Norihiko Nakagawa, Minato-ku, Tokyo (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignee: MITSUI CHEMICALS, INC., Tokyo (JP)

(21) Appl. No.: 11/531,546

(22) Filed: Sep. 13, 2006

(30) Foreign Application Priority Data
Sep. 13, 2005 (JP) 2005-264618

(51) Int. Cl. A61K 9/70; B32B 27/12

(52) U.S. Cl. 424/443; 442/123

(57) ABSTRACT

To provide a fiber assembly in which the durability of antibacterial property, especially the durability of antibacterial property against moisture is improved and there is provided a fiber assembly principally comprising polyolefin, wherein said fiber contains an antibacterial agent and a suspending agent, and has a branched structure.

### BACKGROUND OF THE INVENTION

1. Field of the Invention

The present invention relates to an antibacterial fiber assembly and a production method and uses thereof. More particularly, the present invention relates to an antibacterial fiber assembly having a branched structure and comprising of a particular component.

2. Description of the Related Art

An antibacterial property is imparted to a non-woven fabric from the point of view of the hygiene issue and it is well known in Japanese Patent Laid-Open Publication No. 66108/1999 that a resin composition which can maintain the antibacterial property over a long period of time by using a combination of a compound which imparts the antibacterial agent and hydrophilicity to polyester may be obtained, however, when such resin composition is converted into a fiber, etc., they are inferior inflexibility. Pulp provided with the antibacterial property is also known in Japanese Patent Laid-Open Publication No. 278097/2003, which is inferior in the durability of the antibacterial property. If there exists a non-woven fabric having both flexibility and durability, it is useful. In Japanese Patent Laid-Open Publication No. 21446/2005, a non-woven fabric is described which is formed with a spunbond method combining a specific inorganic antibacterial agent and a surfactant, however the sustainable effect of the antibacterial property cannot be said to be sufficient.

Meanwhile, it is known in Japanese Patent Laid-Open Publication No. 44523/1973 that a non-woven fabric having specific range of a fiber length and fiber diameter and a large strength may be obtained by adding water to a polymer solution in the presence of polyvinyl alcohol, etc. and flashing the mixture.

Patent document 1: Japanese Patent Laid-Open Publication No. 166108/1999

Patent document 2: Japanese Patent Laid-Open Publication No. 278097/2003

Patent document 3: Japanese Patent Laid-Open Publication No. 21446/2005

Patent document 4: Japanese Patent Laid-Open Publication No. 44523/1973

### DISCLOSURE OF THE INVENTION

### Problem to be Solved by the Invention

It is an object of the present invention to provide a fiber assembly in which the durability of the antibacterial property, especially the durability of the antibacterial property against moisture is improved.

### MEANS TO SOLVE PROBLEMS

As a result of earnest studies to solve the aforementioned problems, the present inventors have found that a fiber assembly which has a specific structure and contains an antibacterial agent and a suspending agent may solve the aforementioned problems, and have completed the present invention.

In other words, the present invention relates to a fiber assembly principally comprising polyolefin, wherein said fiber contains an antibacterial agent and a suspending agent, and has a branched structure.

And, the present invention relates to a fiber assembly principally comprising a polyolefin, wherein said fiber contains 0.1 to 30% by mass of an antibacterial agent and 0.1 to 5% by mass of a suspending agent, and having an average fiber length of 0.05 to 50 mm and a branched structure.

Furthermore, the present invention relates to a production method of a fiber assembly having a branched structure, wherein a thermoplastic resin solution is flashed in the presence of water, a suspending agent and an antibacterial agent.

Moreover, the present invention relates to non-woven fabric, filters, packaging materials, cards, sheets and labels, residential materials, hygienic materials and binder fiber as applications of the aforementioned fiber assembly.

### EFFECT OF THE INVENTION

An antibacterial fiber assembly in the present invention has an excellent effect in that it has an excellent antibacterial property and the antibacterial effect is not lost even after long period of use, and further, significantly has an industrial value because said fiber assembly may be formed and converted into a non-woven fabric form which may be used for applications such as filters, packaging materials, cards, sheets and labels, residential materials, hygienic materials, binder fiber, various volatilizers as well as various forming papers.

### BRIEF DESCRIPTION OF THE DRAWINGS

FIG. 1 is a micrograph of a fiber assembly relating to Embodiment 1.

### BEST MODE FOR CARRYING OUT THE INVENTION

Hereinafter, embodiments of the present invention will be explained.

[A Fiber Assembly]

Firstly, a fiber assembly of the present invention is explained. A fiber in the fiber assembly of the present invention comprises principally a polyolefin and contains an antibacterial agent and a suspending agent.

The polyolefin is preferably exemplified by a homopolymer of an α-olefin having 2 to 6 carbon atoms or a copolymer of mutual α-olefins having 2 to 6 carbon atoms; and further a copolymer of α-olefin having 2 to 6 carbon atoms with other copolymerizable olefin, an unsaturated carboxylic acid such as acrylic acid, methacrylic acid, etc., an acrylic ester, a methacrylic ester, vinyl acetate and the like; and furthermore a polymer obtained by graft reacting a homopolymer or a copolymer thereof with an unsaturated carboxylic acid monomer using a peroxide. Especially preferable examples are a crystalline polymer and a copolymer of ethylene, propylene, 1-butene, 3-methyl-1-butene or 4-methyl-1-butene. Specifically, there may be mentioned low-density polyethylene, linear low-density polyethylene and elastomer (ethylene-α-olefin copolymer), medium-densitypolyethylene, high-densitypolyethylene, ultra high molecular weight polyethylene, ethylene-methacrylic acid copolymer; maleic acid or acrylic acid modified-polyethylene, polypropylene, polybutene, poly(3-methylbutene), poly(4-methylbutene); and a mixture thereof. As is clear from the gist of the present invention, these polyolefins may be manufactured by any appropriate method.

Although an antibacterial agent is not specifically limited if it is stable even under heating in manufacturing a fiber assembly, in general an inorganic antibacterial agent is readily used because it is stable against heating. The inorganic antibacterial agent includes a silver-based inorganic antibacterial agent and a non-silver-based inorganic-antibacterial agent. The silver-based inorganic antibacterial agent includes, for example, a silver-supported zeolite (aluminosilicate), silver-supported apatite (calcium phosphate), silver-supported glass (silicon oxide), silver-supported zirconium phosphate, silver-supported calcium silicate, etc. The non-silver-based inorganic antibacterial agent includes, for example, a compound whose active ingredient is zinc oxide, copper oxide, etc., especially a solid solution of aluminum oxide, calcium oxide, magnesium oxide, etc. with zinc oxide or copper oxide, or ultra-fine particles of zinc oxide, titanium oxide, etc.

Although the particle size of the antibacterial agent is not specifically limited, it is preferable that the particle size D_{50%} is in the range of 0.05 to 3 μm from the point of view of the productivity and antibacterial effect of the fiber assembly, and more preferable 0.05 to 2 μm. Here, D_{50%} is a value as measured by laser scattered light after being dispersed with ultrasonic wave for 5 minutes or more.

Although the BET specific surface area of the antibacterial agent is not specifically limited, it is preferable that the BET specific surface area is in the range of 1 to 300 m²/g from the point of view of the antibacterial effect and its durability, and more preferably 10 to 150 m²/g.

The content of the antibacterial agent in the fiber is in the range of 0.1 to 30% by mass, and preferably 0.3 to 10% by mass.

It is considered that the suspending agent reduces the interfacial tension of the fiber and improves the contact efficiency between bacteria and the antibacterial agent, thereby contributing to the improvement of the antibacterial effect.

As suspending agents, there may be used a hydrophilic polymer such as polyvinyl alcohol, polyethylene glycol, polypropylene glycol, polyacrylate salt, gelatin, tragacanth gum, starch, methylcellulose, carboxymethylcellulose, etc. In addition, the hydrophilic polymer may be used in combination with a common nonionic surfactant, cationic surfactant or anionic surfactant. Among them, from the point of view of the productivity and contribution to the antibacterial effect, etc., especially a polyvinyl alcohol-based hydrophilic polymer is preferable and the polymerization degree of the polyvinyl alcohol is preferably in the range of 200 to 1000.

The content of the suspending agent in the fiber is in the range of 0.1 to 5% by mass, and preferably 0.3 to 3% by mass. When the contents of the antibacterial agent and suspending agent fall within the aforementioned range, the fiber assembly obtained exhibits excellent antibacterial property and its durability.

A fiber in the present invention may contain, in addition to the aforementioned antibacterial agent and suspending agent, other components where necessary insofar as the effect of the present invention is not impaired. As other components, there may be mentioned a conventionally known heat resistant stabilizer, weather resistance stabilizer, wide variety of stabilizers, antioxidant, dispersant, antistatic agent, slip agent, anti-blocking agent, anti-clouding agent, lubricating agent, dye, pigment, natural oil, synthetic oil, wax, filling agent, etc.

A fiber in the present invention has an average value of the longest parts of the fiber (referred to as “an average fiber length” in the present description) of 0.05 to 50 mm, and preferably 0.05 to 10 mm, and especially preferably 0.1 to 5 mm. Insofar as the average fiber length falls within the aforementioned range, said fiber is converted into the fiber assembly which may be suitably used for various applications by taking advantage of the antibacterial property.

Moreover, it is preferable that a diameter of a fiber in the present invention (hereinafter, referred to as “a fiber diameter”) has a minimum value of approximately 0.5 μm and a maximum value of approximately 50 μm. Insofar as the fiber diameter falls within the aforementioned range, said fiber is converted into the fiber assembly which may be suitably used for various applications by taking advantage of the antibacterial property.

Here, the methods of measuring the aforementioned average fiber length and fiber diameter will be explained.

(1) Average Fiber Length

Using Automatic Fiber Analyzer manufactured by Metso Automation Inc. in Finland (product name: FiberLab-3.5), a fiber length for 12000 to 13000 filaments of the fiber was measured. Then an average fiber length is recorded as a value obtained by substituting the number average fiber length and the number of filaments of the fiber for each class which are classified in increments of a fiber length of 0.05 mm into the following equation.
Average fiber length(mm)=Σ(Nn×Ln³)/Σ(Nn×Ln²)

Ln: number average fiber length of each class (mm)

Nn: number of filaments of the fiber for each class

Wherein, average fiber length of each class is determined by the following equation.
Ln=ΣL/N

L: measured fiber length of each fiber in one class

N: number of filaments of the fiber in one class

Meanwhile, a fiber length is measured as follows.

A fiber length is measured by dispersing the fiber in water at a dilute concentration, exposing filaments of the fiber passing through the capillary to xenon lamp light to acquire image signals by a CCD (charge-coupled device) sensor and then performing image analysis. More specifically, fibers are dispersed in water at 0.02% by weight, and 12000 to 13000 filaments of the fiber are measured by using Automatic Fiber Analyzer manufactured by Metso Automation Inc. in Finland (product name: FiberLab-3.5). A fiber length is measured in the increment of 0.05 mm and the measurement results of both the fiber length and the presence percentage (%) of the fiber corresponding to each fiber length are obtained.

(2) Fiber Diameter

A fiber diameter is measured by observing each filament of the fiber with an optical microscope or an electron microscope. Specifically, the maximum and minimum values of the fiber diameter are measured as follows.

The maximum value: Filaments of fiber are observed under Digital HF Microscope VH8000 manufactured by Keyence Corporation at a magnification of 100 and 100 parts are randomly selected for parts of 10 μm or more of a diameter and then fiber diameters of the selected parts are measured. The maximum measured value is recorded as the maximum value.

The minimum value: Filaments of fiber are observed under Scanning Electron Microscope JSM6480 manufactured by JEOL Ltd. at a magnification of 3000 and 100 parts are randomly selected for parts of less than 10 μm of a diameter and then fiber diameters of the selected parts are measured. The minimum measured value is recorded as the minimum value.

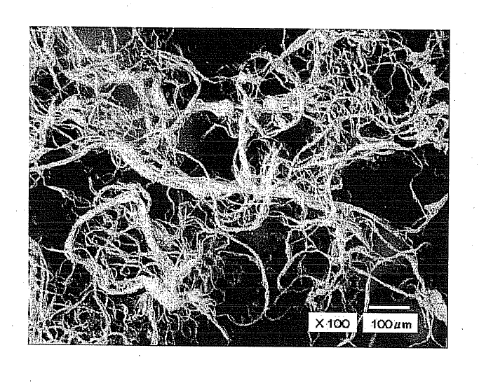

Fiber in the present invention has a branched structure. The branched structure is exemplified by, for example, a configuration illustrated in FIG. 1, wherein a fiber branches into more fine fibers and they are intertwined with each other. Branches of the fiber are confirmed by observing with an optical microscope or an electron microscope. Incidentally, FIG. 1 is a micrograph of the fiber assembly having a branched structure of embodiment 1 described later observed under Digital HF Microscope VH8000 manufactured by Keyence Corporation at a magnification of 100.

In the present invention, a fiber assembly having an average fiber length of 0.05 to 50 mm and a branched structure comprises synthetic pulp which is known as one kind of non-woven fabric and may be manufactured by a flash-spinning method which is one method of manufacturing a non-woven fabric. A flash-spinning method is a process of manufacturing a non-woven fabric by vaporizing the solvent by decompressing the solvent containing the polymer dissolved at a high pressure and further cutting, beating and refining the fiber by a whirling blender, a disc refiner, etc. where necessary. Especially, when an emulsion prepared by dispersing a polyolefin solution in an aqueous medium in the presence of an antibacterial agent and a suspending agent is flashed, by using a method such as described in Japanese Patent Laid-Open Publication No. 44523/1973, there is obtained a fiber assembly of the present invention having an excellent antibacterial property and a configuration in which fiber-like materials are randomly branched. A non-woven fabric (synthetic pulp) formed from such fiber assembly has high strength.

A form of the fiber assembly of the present invention is not specifically limited but includes textile, knit, etc. in addition to a non-woven fabric.

A weight per unit area of the fiber assembly of the present invention may be selected, where necessary, depending on the application.

The fiber assembly of the present invention explained above is excellent in antibacterial property and its durability, especially excellent in durability of the antibacterial property against moisture.

[A Method of Manufacturing a Fiber Assembly]

Next, one embodiment of methods of the present invention of manufacturing a fiber assembly is explained in detail.

A method of manufacturing the fiber assembly of the present invention is a process of flashing a thermoplastic resin solution in the presence of water, a suspending agent and an antibacterial agent, and specifically it is preferable to adopt the following method.

Firstly, an emulsion is prepared by dissolving a raw material resin in a solvent that can dissolve the resin and adding to the resultant solution the aforementioned suspending agent, antibacterial agent and water. As the raw material resin, the aforementioned polyolefin is suitable.

Solvent may be selected appropriately from those which dissolve raw material resin and are less likely to remain in the obtained fiber assembly when vaporized by flashing, including saturated hydrocarbon based solvents such as butane, pentane, hexane, heptane, octane, cyclohexane, etc.; aromatic based solvents such as benzene, toluene, etc.; halogenated hydrocarbons such as methylene chloride, chloroform, carbon tetrachloride, etc.; and the like.

It is preferable that the amount of the added suspending agent is set to the amount by which the concentration of the suspending agent results in 0.1 to 5% by mass in the fiber. When an added amount of polyvinyl alcohol as suspending agent is set to the amount by which the concentration of the suspending agent results in less than 0.1% by mass, resultant instability of emulsion causes fiber thick and the maximum diameter of fiber results in 80 μm. And stable cutting of the fiber is prevented as the fiber is floated on the surface of a water and cannot be dispersed in the water. On the contrary when an amount of poly vinyl alcohol exceeds the concentration of 5% by mass, a dehydration rate becomes very slow at a process of making a paper with a mixture of other fibers such as a natural pulp and this causes an adverse influence on an efficient product manufacture.

In the manufacturing process, when an operation in which part of the added suspending agent is removed is performed, the suspending agent is added by adjusting accordingly, for example, adding somewhat excessively. A target of the amount of the added suspending agent is 0.1 to 10 parts by mass, based on 100 parts by mass of a raw material resin. The addition of the suspending agent enables to stabilize the emulsion as well as to perform stably the fiber cutting in the water after flashing.

While, it is preferable that an amount of the added antibacterial agent is set to the amount by which the concentration of antibacterial agent results in 0.1 to 30% by mass in the fiber. In a manufacturing process, when an operation in which part of the added antibacterial agent is removed is performed, the antibacterial agent is added by adjusting accordingly, for example, adding somewhat excessively. A target of the amount of the added antibacterial agent is 0.1 to 50 parts by mass, based on 100 parts by mass of the raw material resin.

Further, preferable amount of the added antibacterial agent varies with the application of the fiber assembly obtained. For example, for a water purification filter which is used over a long period of time, the amount of the added antibacterial agent is approximately 1 to 3 parts by mass, based on 100 parts by mass of the raw material resin. Even for the same water purification filter, when it is mixed with other material such as activated carbon, it is preferable, in order to secure the absolute amount of the antibacterial agent in the filter, that the amount of the added antibacterial agent is approximately 1 to 30 parts by mass, based on 100 parts by mass of the raw material resin.

Next, the emulsion obtained is heated to a temperature between 100 and 200° C., preferably between 130 and 150° C. and compressed to a pressure between 0.1 and 5 MPa, preferably between 0.5 and 1.5 MPa, and ejected (flashed) to a reduced pressure from a nozzle, whereby the solvent is simultaneously evaporated. It is preferable that the conditions of reduced pressure are 1 kPa to 95 kPa and the space to which the emulsion is ejected is filled with an inert atmosphere such as a nitrogen atmosphere, etc.

Here, in the present description, pressure represents the absolute pressure.

Fiber of undefined length having a branched structure is obtained by flashing the emulsion as mentioned above. However, it is preferable that the fiber is processed to a desired length by cutting, beating and refining using a whirling blender, a disc refiner, etc. In this case, it is preferable to perform the above cutting, beating and refining processing by preparing an aqueous slurry containing the fiber at the concentration of 0.5 to 5 g/l. After drying, the resultant fiber may be opened as desired by a mixer, etc.

According to the method explained above, a fiber assembly having a branched structure, especially a fiber assembly of the present invention may be preferably manufactured.

[Applications of a Fiber Assembly]

A fiber assembly of the present invention has an excellent antibacterial property and has an excellent effect that the antibacterial effect is not impaired even after a long period of use, especially after a long period of use in the presence of moisture.

Such fiber assembly may be suitably used by forming, for example, into a non-woven fabric for various applications including filters such as tea bag paper, coffee bag paper, soup package paper, air filter, mask, water purification filter, wine filter, beer filter, juice filter, etc.; packaging materials such as food packaging paper, deoxidizer packaging paper, medical packaging paper, insect resistant packaging paper, etc.; cards, sheets and labels such as wall paper, moisture-permeable and waterproof sheet, heat resistant board, fusuma paper, shoji paper, greeting card, pamphlet, business card, book cover, envelope, lamp shade, label paper, printing paper, poster paper, etc.; residential materials such as trapping agent of cement particles, thixotropy imparting agent, etc.; hygienic materials such as top sheet and absorbent binder fiber of disposable diaper, napkin and sheet, binder fiber of disposable hand towel, wiper and tissue, oil blotting paper, sterilizing paper, etc.; vaporizer of steam vaporizer for humidifier, fragrance core, etc.; and binder fiber of food tray, writing materials, buffer for large components, door panel of car, etc.

Moreover, the materials in the above applications may be composed of only a fiber assembly of the present invention, or other fibers may be combined with the fiber assembly of the present invention.

Hereinafter, the present invention will be explained more specifically with reference to embodiments, etc., but the range of the present invention is not restricted to these embodiments, etc.

### EMBODIMENT 1

[A Method of Manufacturing a Fiber Assembly]

Into an 80 L autoclave equipped with a stirrer were charged 20 L of n-hexane (23° C.), 20 L of water (23° C.), 1 kg of polyethylene (Hizex 2200J, manufactured by Mitsui Chemicals, Inc.; melting point 135° C.), 20 g of polyvinyl alcohol (Gohsenol NL-05, manufactured by Nippon Synthetic Chemical Industry Co., Ltd.) and 6 g of an antibacterial agent (Seabio Z-28, manufactured by Research Institute for Oceanochemistry). The liquid mixture was heated to 145° C. with stirring. Stirring was continued for 30 min. by maintaining the temperature at 145° C. to obtain an emulsion.

Then, the emulsion was flashed into a drum maintained in a nitrogen atmosphere at a pressure of 53 kPa via a nozzle having a diameter of 3 mm and a length of 20 mm mounted on the autoclave, and a fiber-like material was obtained.

Next, after preparing an aqueous slurry containing the fiber-like material at a concentration of 10 g/L, the slurry was beaten and refined in a 12-inch diameter disc-type refiner to obtain a fiber assembly dispersed in water. The dispersed fiber assembly was dried using a hot air circulation dryer at 50° C. for 24 hours and the resultant fiber assembly was opened in a 2 L household mixer to obtain a flocculent fiber assembly.

The obtained fiber assembly had a branched structure in fiber structure as illustrated in FIG. 1. FIG. 1 is a micrograph of the fiber assembly after drying observed under Digital HF Microscope VH8000 manufactured by Keyence Corporation at a magnification of 100.

The fiber assembly had a fiber diameter distribution in which the minimum and maximum values were 1.1 μm and 38 μm, respectively, and a fiber length distribution ranging from 0.1 mm to 5 mm. The average fiber length was 1.21 mm. The content of the antibacterial agent in the fiber was 0.49% by mass and the content of polyvinyl alcohol in the fiber was 1.9% by mass.

[Evaluation Method of an Antibacterial Property]

For the flocculent fiber assembly prepared by the above method, a bacteriostatic activity value was determined by JIS L1902 absorption method for emulsion of bacteria (test for antibacterial activity and efficacy approved by Japan Textile Evaluation Technology Council) and an antibacterial capability was evaluated.

(1) A Preparation Method of Test Sample Used for the Evaluation

Into a metal receptacle of 18 mm×18 mm was placed 0.4 g of the flocculent fiber assembly, and the fiber assembly was uniformly spread, and compressed at a pressure of 63.7 MPa for 10 minutes to prepare a 18 mm-square test specimen weighing 0.4 g.

(2) Pretreatment of Test Samples

The test sample prepared by the above method was placed in a vial bottle and sterilization by high-pressure steam was performed at a temperature of 121° C. and pressure of 103 kPa for 15 minutes in an autoclave.

(3) Culture Test

The sterilized test sample placed in a vial bottle was inoculated uniformly as much as possible with 0.2 mL bacteria emulsion of a Staphylococcus aureus ATCC 6538P (Staphylococcus aureus) whose vial count was controlled to (1±0.3)×105 and was cultured at 37° C. for 18 hours. To the culture medium was added 20 mL of physiological saline added with 0.2% of Tween 80 and the mixed solution was stirred to wash out the bacteria. A 10-fold dilution series of the washed-out bacteria was prepared and carried out pour culture with a nutrient agar culture medium at 37° C. for 24 hours or more to count the number of colonies and determine the viable count.

(4) Calculation of Test Results

For a standard sample and the test samples, each of the above tests was performed to determine the bacteriostatic activity value from the following equation. A standard white cotton fabric described in JIS L0803 was used as a standard sample.
Bacteriostatic activity value=log B−log C

B=Viable count after incubation for 18 hours in the presence of the standard sample

C=Viable count after incubation for 18 hours in the presence of the test sample

Here, a bacteriostatic activity value of 2.2 or more was judged to be good and marked AA in Table 1. In addition, a bacteriostatic activity value less than 2.2 was judged to be poor and marked BB in Table 1.

(5) Evaluation of Durability

Into a 20 L vessel equipped with a cover were charged 20 L of tap water and a flocculent antibacterial sample for evaluation prepared by the above (1) and the liquid mixture was vigorously shaken and allowed to settle for one day. The sample for evaluation was recovered by filtering through a mesh, and dried at 50° C. for one day. The antibacterial evaluation was performed for such resultant samples in accordance with the description in the above items (2) to (4).

The results are shown in Table 1. As can be seen from Table 1, the sample of Embodiment 1 exhibited good bacteriostatic activity values both immediately after manufacturing and one day after immersion in tap water, and exhibited that the sample has durability of the antibacterial property as well as antibacterial property.

[Analytical Method of the Content of Antibacterial Agent]

A film having a thickness of 1 mm is prepared by sandwiching a flocculent fiber assembly together with a metal spacer having a thickness of 1 mm between films made of a fluorocarbon resin under the conditions of a constant temperature of 25° C. and a constant humidity of 50%, followed by fusing the fiber assembly by pressing at 180° C. for 3 minutes. The film is cut into about 1 cm square pieces and 10 g thereof is put in a porcelain crucible. The pieces are burned in an electric furnace at 1100° C. and the weight of the residue is weighed. The content of antibacterial agent in the fiber is determined by the following equation as a percentage.
The content of antibacterial agent(%)=(Residue after burning(g)/10(g))×100
[Quantification Method of Polyvinyl Alcohol]

A film having a thickness of approximately 200 μm is prepared by sandwiching a flocculent fiber assembly together with a metal spacer having a thickness of 200 μm between films made of a fluorocarbon resin under the conditions of a constant temperature of 25° C. and a constant humidity of 50%, followed by fusing the fiber assembly by pressing at 180° C. for 3 minutes. Absorbance of the film at 1100 cm⁻¹ is measured by an infrared absorption spectrum analyzer (FT/IR-400, manufactured by JASCO Corporation). The film thickness is measured in the unit of μm. Content of polyvinyl alcohol is determined from the measured film thickness (μm) and the absorbance using Lambert-Beer's law.

In addition, in determining the content, a polyethylene sample of the same form containing 2.0% of polyvinyl alcohol was prepared in advance and used as a standard sample. The standard sample was prepared by adding 2 parts by mass of polyvinyl alcohol to 98 parts by mass of polyethylene, followed by fusing and kneading.

### EMBODIMENT 2

A fiber assembly was prepared by the same method as Embodiment 1 by changing the type of antibacterial agent described in Embodiment 1 to Novalon AG1100 manufactured by Toagosei Co., Ltd.

The obtained fiber assembly had a branched structure in fiber structure. The fiber assembly had a fiber diameter distribution in which the minimum and maximum values were 1.2 μm and 35 μm, respectively, and a fiber length distribution ranging from 0.1 mm to 5 mm. An average fiber length was 1.19 mm. A content of the antibacterial agent in the fiber was 0.46% by mass and a content of polyvinyl alcohol in the fiber was 1.9% by mass.

The fiber assembly was evaluated for antibacterial property by the same method as Embodiment 1. The results are shown in Table 1.

As can be seen from Table 1, like the sample of embodiment 1, the sample of Embodiment 2 exhibited good bacteriostatic activity values both immediately after manufacturing and one day after immersion in tap water, and exhibited that the sample has durability of the antibacterial property as well as antibacterial property.

### EMBODIMENT 3 AND 4

A fiber assembly was prepared by the same method as Embodiment 1 by changing the amount of antibacterial agent added from 0.6 part by mass to 0.1 part by mass and 30 parts by mass.

The obtained fiber assembly had a branched structure in fiber structure and its evaluated physical property and antibacterial property are shown in Table 1. As can be seen from Table 1, like the sample of embodiment 1, the samples of Embodiment 3 and 4 exhibited good bacteriostatic activity values both immediately after manufacturing and one day after immersion in tap water, and exhibited that the sample has durability of the antibacterial property as well as antibacterial property.

### COMPARATIVE EMBODIMENT 1

A core-sheath composite fiber having a core/sheath ratio=50/50 was prepared by melt-spinning through a spinning nozzle, wherein the sheath component was composed of 100 parts by mass of polyethylene (Hizex 2200J, manufactured by Mitsui Chemicals, Inc.) to which 0.6 part by mass of an antibacterial agent Novalon AG1100 and 2 parts by mass of polyvinyl alcohol were admixed and the core component was composed of polypropylene (Hipol, manufactured by Mitsui Chemicals, Inc.). After preparing long fiber with 2 deniers by extending the composite fiber, it was cut into 5 mm long pieces to obtain short fiber with the fiber diameter of 25 μm without having a branched structure. A content of the antibacterial agent in the fiber was 0.30% and that of polyvinyl alcohol in the fiber was 1.0%.

Using the above core/sheath ratio, the content of the antibacterial agent in the sheath side was calculated to be 0.60% and the content of polyvinyl alcohol in the sheath side was calculated to be 2.0%.

The short fiber was evaluated for antibacterial property by the same method as Embodiment 1. The results are shown in Table 1.

It was found from Table 1 that the sample of Comparative Embodiment 1 exhibited a good bacteriostatic activity value immediately after manufacturing, however, the sample had a decreased bacteriostatic activity value one day after immersion in tap water and had no durability of the antibacterial property.

### COMPARATIVE EMBODIMENT 2

A fiber assembly was prepared by the same method as Embodiment 1 without adding an antibacterial agent. The obtained fiber assembly had a branched structure in fiber structure. The fiber assembly had a fiber diameter distribution in which the minimum and maximum values were 1.1 μm and 34 μm, respectively, and a fiber length distribution from 0.1 mm to 5 mm. The average fiber length was 1.20 mm. The amount of polyvinyl alcohol in the fiber was 1.8% by mass.

The fiber assembly was evaluated for antibacterial property by the same method as Embodiment 1. The results are shown in Table 1.

It was found from Table 1 that the obtained fiber assembly has no antibacterial property.

### COMPARATIVE EMBODIMENT 3

A fiber assembly was prepared by the same method as Embodiment 1 by changing the type of antibacterial agent described in Embodiment 1 to Zeomic (SINANEN ZEOMIC (registered trademark))

The obtained fiber assembly had a branched structure in fiber structure. Its evaluated physical property and antibacterial property are shown in Table 1. As can be seen from Table 1, the samples of Comparative Embodiment 3 exhibited poor antibacterial property.

TABLE 1

| | | | | | Comparative | Comparative | Comparative |
|---|---|---|---|---|---|---|---|
| | Embodiment 1 | Embodiment 2 | Embodiment 3 | Embodiment 4 | Embodiment 1 | Embodiment 2 | Embodiment 3 |
| Synthetic resin | 100 parts of | 100 parts of | 100 parts of | 100 parts of | 100 parts of HDPE | 100 parts of | 100 parts of HDPE |
| | HDPE¹⁾ | HDPE | DPE | HDPE | (sheath side)/ | HDPE | |
| | | | | | 100 parts of PP²⁾ | | |
| | | | | | (core side) | | |
| Antibacterial agent | 0.6 part of | 0.6 part of | 0.1 parts of | 30 parts of | 0.6 part of Novalon | — | 0.6 part of Zeomic |
| | Seabio | Novalon | Novalon | Novalon | (in the sheath | | |
| | | | | | side.) | | |
| Fiber spinning method | Flash spinning | Flash spinning | Flash spinning | Flash spinning | Melt-spinning | Flash spinning | Flash spinning |
| | method | method | method | method | method | method | method |
| Fiber structure | Multi-branched | Multi-branched | Multi-branched | Multi-branched | No branched | Multi-branched | Multi-branched |
| | | | | | structure | | |
| Fiber diameter (μm) | 1.1 | 1.2 | 1.2 | 1.2 | 25 | 1.1 | 1.2 |
| Minimum value | | | | | | | |
| Fiber diameter (μm) | 38 | 35 | 35 | 35 | 25 | 34 | 35 |
| Maximum value | | | | | | | |
| Fiber length (mm) | 0.1˜5 | 0.1˜5 | 0.1˜5 | 0.1˜5 | 5 | 0.1˜5 | 0.1˜5 |
| Average fiber length | 1.21 | 1.19 | 1.23 | 1.21 | 5 | 1.20 | 1.19 |
| (mm) | | | | | | | |
| Content of | 0.49 | 0.46 | 0.098 | 25 | 0.30 | — | 0.28 |
| antibacterial agent | | | | | | | |
| (%; Analytical value) | | | | | | | |
| (Ana. V)³⁾/ | 81 | 76 | 98 | 64 | | | 46 |
| (Add. V)⁴⁾ * 100 | | | | | | | |
| Content of polyvinyl | 1.9 | 1.9 | 1.9 | 1.9 | 1.0 | 1.8 | 1.9 |
| alcohol | | | | | | | |
| (%; analytical value) | | | | | | | |
| Bacteriostatic activity | more than 4.3 | more than 4.3 | 2.3 | more than 4.3 | more than 4.3 | 1.3 | 2.0 |
| value immediately | | | | | | | |
| after manufacturing | | | | | | | |
| Bacteriostatic | AA⁵⁾ | AA | AA | AA | AA | BB⁶⁾ | BB |
| activity evaluation | | | | | | | |
| immediately after | | | | | | | |
| manufacturing | | | | | | | |
| Bacteriostatic | more than 4.3 | more than 4.3 | 2.3 | more than 4.3 | 0.8 | 1.3 | 2.0 |
| activity value | | | | | | | |
| one day after | | | | | | | |
| immersion in | | | | | | | |
| tap water | | | | | | | |
| Bacteriostatic | AA | AA | AA | AA | BB | BB | BB |
| activity evaluation | | | | | | | |
| one day after | | | | | | | |
| immersion in tap water | | | | | | | |

¹⁾HDPE: high density polyethylene
²⁾PP: polypropylene
³⁾Ana. V: Analytical value of antibacterial agent content
⁴⁾Add. V: Amount of antibacterial agent added
⁵⁾AA: good
⁶⁾BB: poor

### INDUSTRIAL APPLICABILITY

A fiber assembly of the present invention is suitable for non-woven fabric, filters, packaging materials, cards/sheets/labels, residential materials, hygienic materials, binder fiber, etc. because of the excellent antibacterial property and durability of the antibacterial property against moisture.

What is claimed is:

1. A fiber assembly principally comprising polyolefin, wherein said fiber contains an antibacterial agent and a suspending agent, and has a branched structure.

2. The fiber assembly according to claim 1 principally comprising polyolefin, wherein said fiber contains 0.1 to 30% by mass of an antibacterial agent and 0.1 to 5% by mass of a suspending agent, and has an average fiber length of 0.05 to 50 mm and a branched structure.

3. The fiber assembly according to claim 1, wherein said suspending agent is polyvinyl alcohol and said antibacterial agent is an inorganic compound.

4. The fiber assembly according to claim 1, wherein said fiber assembly is formed by a flash spinning method.

5. A method of manufacturing a fiber assembly having a branched structure, wherein a thermoplastic resin solution is flashed in the presence of water, a suspending agent and an antibacterial agent.

6. A non-woven fabric obtained by forming the fiber assembly according to claim 1.

7. A filter containing the fiber assembly according to claim 1.

8. A packaging material containing the fiber assembly according to claim 1.

9. A card containing the fiber assembly according to claim 1.

10. A sheet containing the fiber assembly according to claim 1.

11. A label containing the fiber assembly according to claim 1.

12. A residential material containing the fiber assembly according to claim 1.

13. A hygienic material containing the fiber assembly according to claim 1.

14. A binder fiber containing the fiber assembly according to claim 1.
